SEQUENCE LISTING (26 sequences)

SEQ ID NO: 1 - DNA, 2228 bp, Homo sapiens
cttaagagcgcggccggaaagcagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagggaatactactatggatataaaagatctggttaaacaatttcatcagattaaagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttaggtaaagtttcatctattcattctgatcaatctgatgtgttaatggtggatgtggcaaccagtatgcctgttgttatcaaggaggtgccaagctcagaggctgctgaagatgtcatggtggctgctcctcttgtgtgttctggaaaggtgctagaagtgcaggttgtgaaccaaacatctgtccatgtagatttgggttctcaacctaaagaaaatgaggctgaaccatcaacggcttcctcagtccccttgcaggatgaacaagaacctcctgcttatgatcaagctcctgaggtcactttgcaggctgatattgaggttatgtcaactgttcatatatcatctgtctataacgatgtgcctgtgactgaaggagttgtttatatcgagcaactgccagaacaaatagttatcccttttactgatcaagttgcttgtcttaaagaaaatgagcagtcaaaagaaaatgagcagtcaccacgagttagtcccaaatctgtagtagaaaagaccacctctggcatgtctaaaaaatctgtagagtccgtaaaacttgcacagttggaggagaatgcaaaatattcctcagtatatatggaggcagaagcaacagctctgctctctgacacatctttgaaaggtcagcctgaggtacctgcacaactcctggatgcagaaggtgctatcaaaataggctctgaaaaatctctgcaccttgaagtggaggtcacttcaatagtctctgacaatactgggcaggaggagtctggggaaaactctgtaccccaggagatggaaggcagacctgtgctctctggggaagctgcagaagcagtgcactcaggtacatctgtaaagtcatctagtggccccttccctcctgctccagaaggccttactgcaccagaaattgaaccagaaggggaatcaacagctgaataaggtttgatgaagccagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcccggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgcaagctt

SEQ ID NO: 2 - protein, 697 aa, Homo sapiens
MISSKPRLVVPYGLKTLLEGISRAVLKTNPSNINQFAAAYFQELTMYRGNTTMDIKDLVKQFHQIKVEKWSEGTTPQKKLECLKEPGKTSVESKVPTQMEKSTDTDEDNVTRTEYSDKTTQFPSVYAVPGTEQTEAVGGLSSKPATPKTTTPPSSPPPTAVSPEFAYVPADPAQLAAQMLGKVSSIHSDQSDVLMVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAEGLMKPAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSDSGDKCAPFGSYGIAGEVTVTTAHKRRKAETEN

SEQ ID NO: 3 - protein, 599 aa, Homo sapiens
MEKSTDTDEDNVTRTEYSDKTTQFPSVYAVPGTEQTEAVGGLSSKPATPKTTTPPSSPPPTAVSPEFAYVPADPAQLAAQMLGKVSSIHSDQSDVLMVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAEGLMKPAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSDSGDKCAPFGSYGIAGEVTVTTAHKRRKAETEN

SEQ ID NO: 4 - protein, 519 aa, Homo sapiens
MLGKVSSIHSDQSDVLMVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAEGLMKPAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSDSGDKCAPFGSYGIAGEVTVTTAHKRRKAETEN

SEQ ID NO: 5 - protein, 503 aa, Homo sapiens
MVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAEGLMKPAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSDSGDKCAPFGSYGIAGEVTVTTAHKRRKAETEN

SEQ ID NO: 6 - protein, 496 aa, Homo sapiens
MPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAEGLMKPAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSDSGDKCAPFGSYGIAGEVTVTTAHKRRKAETEN

SEQ ID NO: 7 - protein, 14 aa, Homo sapiens
LKTLLEGISRAVLK

SEQ ID NO: 8 - protein, 15 aa, Homo sapiens
VSDNTGQEESGENSV

SEQ ID NO: 9 - protein, 11 aa, Homo sapiens
SGTSVKSSSGR

SEQ ID NO: 10 - protein, 11 aa, Homo sapiens
NQFAAAYFQEL

SEQ ID NO: 11 - protein, 10 aa, Homo sapiens
VEKWSEGTTP

SEQ ID NO: 12 - protein, 13 aa, Homo sapiens
KTTQFPSVYAVPG

SEQ ID NO: 13 - protein, 16 aa, Homo sapiens
PSSPPPTAVSPEFAYV

SEQ ID NO: 14 - protein, 16 aa, Homo sapiens
AEATALLSDTSLKGQP

SEQ ID NO: 15 - protein, 292 aa, Homo sapiens
MPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAE

SEQ ID NO: 16 - protein, 204 aa, Homo sapiens
GLMKPAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSDSGDKCAPFGSYGIAGEVTVTTAHKRRKAETEN

SEQ ID NO: 17 - protein, 8 aa, Homo sapiens
LVVPYGLK

SEQ ID NO: 18 - protein, 8 aa, Homo sapiens
TLLEGISR

SEQ ID NO: 19 - protein, 21 aa, Homo sapiens
TNPSNINQFAAAYFQELTMYR

SEQ ID NO: 20 - protein, 20 aa, Homo sapiens
KYSSVYMEAEATALLSDTSL

SEQ ID NO: 21 - protein, 16 aa, Homo sapiens
GQPEVPAQLLDAEGAI

SEQ ID NO: 22 - DNA, 26 bp, Artificial Sequence
  A degenerate sense primer used to isolate the CBP86 cDNA
ggncagccngaggtnccngcncaryt

SEQ ID NO: 23 - protein, 9 aa, Homo sapiens
GQPEVPAQL

SEQ ID NO: 24 - DNA, 39 bp, Artificial Sequence
  An antisense 3′ gene-specific primer for CBP86
ttattcagctgttgattccccttctggttcaatttctgg

SEQ ID NO: 25 - DNA, 879 bp, Homo sapiens
atgcctgttgttatcaaggaggtgccaagctcagaggctgctgaagatgtcatggtggctgctcctcttgtgtgttctggaaaggtgctagaagtgcaggttgtgaaccaaacatctgtccatgtagatttgggttctcaacctaaagaaaatgaggctgaaccatcaacggcttcctcagtccccttgcaggatgaacaagaacctcctgcttatgatcaagctcctgaggtcactttgcaggctgatattgaggttatgtcaactgttcatatatcatctgtctataacgatgtgcctgtgactgaaggagttgtttatatcgagcaactgccagaacaaatagttatcccttttactgatcaagttgcttgtcttaaagaaaatgagcagtcaaaagaaaatgagcagtcaccacgagttagtcccaaatctgtagtagaaaagaccacctctggcatgtctaaaaaatctgtagagtccgtaaaacttgcacagttggaggagaatgcaaaatattcctcagtatatatggaggcagaagcaacagctctgctctctgacacatctttgaaaggtcagcctgaggtacctgcacaactcctggatgcagaaggtgctatcaaaataggctctgaaaaatctctgcaccttgaagtggaggtcacttcaatagtctctgacaatactgggcaggaggagtctggggaaaactctgtaccccaggagatggaaggcagacctgtgctctctggggaagctgcagaagcagtgcactcaggtacatctgtaaagtcatctagtggccccttccctcctgctccagaaggccttactgcaccagaaattgaaccagaaggggaatcaacagctgaataa

SEQ ID NO: 26 - DNA, 600 bp, Homo sapiens
gcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcccggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactga

(19) United States
(12) Patent Application Publication
(10) Pub. No.: US 2003/0207393 A1
(43) Pub. Date: Nov. 6, 2003

(54) Cbp86, a sperm specific protein

(76) Inventors: John C. Herr, Charlottesville, VA (US); Sen Buer, Lilburn, GA (US); Arabinda Mandal, Charlottesville, VA (US); Michael Wolkowicz, Charlottesville, VA (US); Soren Naaby-Hansen, London (GB)

Correspondence Address: John P Breen, University of Virginia Patent Foundation, Suite 1 110, 1224 West Main Street, Charlottesville, VA, 22903, US

(21) Appl. No.: 10/181,638

(22) PCT Filed: Jan. 19, 2001
(86) PCT No.: PCT/US01/01715

(51) Int. Cl. C07K 14/435; C12P 21/02; C12N 5/06; C07H 21/04; C07K 16/18

(52) U.S. Cl. 435/69.1; 435/320.1; 435/325; 530/350; 530/388.1; 536/23.5

(57) ABSTRACT

The present invention relates to acidic (pI 4.0) 86 kDA isoforms of a novel, polymorphic, testis-specific protein designated calcium binding protein 86 (CBP86). This protein is tyrosine phosphorylated during in vitro capacitation and bound calcium⁴⁵ on 2-D gels, the latter effect abolished by dephosphorylation with alkaline phosphatase. CBP86 localizes to the principal piece of the human sperm flagellum in association with the fibrous sheath and is the first demonstration of a sperm protein that both oligomerizes and gains calcium binding capacity in a tyrosine phosphorylation dependent manner during capacitation.

This application claims priority under 35 U.S.C. §119(e) to provisional patent application No. 60/176,887, filed Jan. 19, 2000.

### US GOVERNMENT RIGHTS

This invention was made with United States Government support under Grant No. HED U54 29099, awarded by the National Institutes of Health. The United States Government has certain rights in the invention.

### FIELD OF THE INVENTION

The present invention is directed to acidic (pI 4.0) 86 kDa isoforms of a novel, polymorphic, testis-specific protein, designated calcium binding protein 86 (CBP86). This protein is tyrosine phosphorylated during in vitro capacitation and binds calcium after being phosphorylated.

### BACKGROUND OF THE INVENTION

Fertilization capacity is acquired by spermatozoa only after residence in the distinct microenvironments of the uterus or oviduct (depending on the species) for a finite period of time. The necessary series of changes, termed capacitation, was first described independently by Chang and Austin in the early to mid 1950s. Capacitation involves molecular changes in both the sperm head and tail which allow defined physiological endpoints to occur such as motility hyperactivation, a whiplash-like sperm tail motion, and regulated acrosomal exocytosis. Hyperactivation is observed when sperm reach the oocyte and increase their flagellar bend amplitude and beat asymmetry which are thought to enhance the ability of sperm to penetrate the egg vestments by increasing forward progression and lateral flagellar thrust.

Our understanding of the molecular mechanisms underlying capacitation and hyperactivation is rudimentary at present but there is evidence that Ca²⁺, cAMP and protein tyrosine phosphorylation are involved. Capacitation can be accomplished in vitro using cauda epididymal or ejaculated sperm incubated in defined media containing a protein source such as albumin, NaHCO₃, Ca²⁺ and energy substrates such as glucose, pyruvate or lactate. Conditions conducive to in vitro capacitation lead to increased tyrosine phosphorylation of a subset of proteins in both mouse and human sperm. The removal of albumin, NaHCO₃, or Ca²⁺ from capacitation media prevents the occurrence of both tyrosine phosphorylation and capacitation. Two protein substrates for this capacitation-related phosphorylation are members of the A kinase anchoring protein family, AKAP 4 (originally called AKAP82 or Fsc 1 in mouse) and AKAP3 (originally called AKAP95T, FSP95 or AKAP110), which are components of the fibrous sheath of the sperm tail.

Although little is known about the kinetics of intracellular calcium during capacitation, a massive influx of Ca²⁺ occurs during the acrosome reaction, and extracellular calcium is required for sperm hyperactivation. If hyperactivated sperm are transferred to calcium free media for 30-60 min, none are hyperactive, but hyperactivation can be restored by addition of 2 mM calcium. Calcium is also known to increase flagellar bend amplitude in demembranated sperm. Intracellular calcium [Ca²⁺ᵢₙ] is increased in hyperactivated sperm in both the head and tail, and Ca²⁺ᵢₙ oscillates with each flagellar bend, indicating a direct relationship between intracellular calcium and hyperactivation.

The cytosolic level of cAMP increases during capacitation, and pharmacological stimulants which elevate intracellular cAMP such as the phosphodiesterase inhibitors, caffeine and pentoxifylline enhance sperm hyperactivated motility, enhance penetration of cervical mucus, increase tight binding to homologous zona pellucida, and increase fertilization. Calcium/calmodulin is an activator of both mammalian sperm adenylate cyclase (AC) and cyclic nucleotide phosphodiesterase, and sperm AC is stimulated by HCO₃⁻ anions. A soluble testicular adenylate cyclase has recently been cloned and shown to be sensitive to bicarbonate. Sperm protein tyrosine phosphorylation is accelerated by cAMP agonists, while antagonists of PKA inhibit tyrosine phosphorylation and capacitation. These and other observations suggest that sperm protein tyrosine phosphorylation and capacitation are under the regulation of a cAMP/PKA pathway, which is activated by elevated cytosolic levels of calcium and HCO₃⁻ anions. Mammalian sperm contain all three subtypes of the guanine nucleotide-binding regulatory proteins Gᵢ, and G proteins have been localized in particular to the sperm tail where protein kinase A and C have also been reported. A cyclic nucleotide gated Ca²⁺ channel in mammalian sperm plasma membranes has been reported, and N- and R-type Ca²⁺ channels have been defined in mouse sperm.

The present invention is directed to targets at the intersection between the calcium and protein tyrosine kinase signal transduction pathways in human spermatozoa. In particular, the present invention describes the isolation and characterization of a sperm calcium binding protein that is also phosphorylated by tyrosine kinases.

Definitions

In describing and claiming the invention, the following terminology will be used in accordance with the definitions set forth below.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;

3. peptides wherein the C terminus is derivatized to —C(O)R₂ where R ₂ is selected from the group consisting of C₁-C₄ alkoxy, and —NR₃R₄ where R₃ and R₄ are independently selected from the group consisting of hydrogen and C₁-C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Vat or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for trytophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” are defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met Leu, Ile, Val, Cys

V. Large, aromatic residues:

Phe, Tyr, Trp

As used herein, the term “purified” and like terms relate to the isolation of a molecule or compound in a form that is substantially free of contaminants normally associated with the molecule or compound in a native or natural environment.

As used herein, the term “CBP86 polypeptide” and like terms refers to polypeptides comprising SEQ ID NO: 2 and biologically active fragments thereof.

As used herein, the term “biologically active fragments” or “bioactive fragment” of an CBP86 polypeptide encompasses natural or synthetic portions of SEQ ID NO: 2 that are capable of specific binding to at least one of the natural ligands of the native CBP86 polypeptide.

“Operably linked” refers to a juxtaposition wherein the components are configured so as to perform their usual function. Thus, control sequences or promoters operably linked to a coding sequence are capable of effecting the expression of the coding sequence.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

### SUMMARY OF THE INVENTION

The present invention is directed to the isolation and characterization of a novel testis and sperm-specific, calcium binding protein, CBP86, that is expressed post-meiotically and localized in the sperm flagellum. This protein exhibits increased tyrosine phosphorylation during in vitro capacitation and increased calcium binding isoforms during capacitation.

### BRIEF DESCRIPTION OF THE DRAWINGS

FIG. 1 is schematic representation of the potential translational variants of CBP86. Twelve predicted CBP86 forms are indicated by Roman numerals (I through XII, respectively). Forms I-V are predicted through alternative start sites and readthrough between amino acids 493 and 499. Splice variants VII, VIII and X-XII, indicated by asterisks, were cloned and sequenced from cDNA libraries. Clone VI was initially amplified from human testicular adaptor-ligated cDNA and was verified by cDNA library cloning. The predicted number of amino acids, pI's and molecular weights (MW) as well as the observed MW calculated from reduced and carboxymethylated sperm peptides, are noted for each form. The coding regions of each of the CBP86 variants are shown as blocked regions. The stippled region of variant XI indicates a sequence not found in any other CBP86 cDNA sequence. The crosshatched region of variants I-V represents the readthrough region. Splice junctions found in each variant are numbered and the contiguous amino acid sequences at the beginning and end of the splice sites are noted below each junction.

FIG. 2A shows a multiple tissue Northern Blot, wherein CBP86 cDNA corresponding to CR-A was radiolabeled with P³² and hybridized to 2 ug poly-(A)+ mRNAs, revealing 2.4 and 1.4 Kb messages only in testicular RNA. Size of molecular weight markers is indicated at left, lanes 1-8 contain poly-(A)+ mRNA isolated from spleen, thymus, prostate, testis, ovary, small intestine, colon and leucocyte, respectively. The lower panel of FIG. 2A shows the identical blot probed with β-actin cDNA as a positive control.

FIG. 2B shows a dot-blot tissue-mRNA Northern probed with P³²-labeled CBP86 cDNA revealed hybridization only in testis (D1). The normalized (100-500 ng) poly-(A)+ mRNAs present on the grid were isolated from various tissue sources: A 1-8 represents whole brain, amygdala, caudate nucleus, cerebellum, cerebral cortex, frontal lobe, hippocampus, medulla oblongata, respectively; B 1-7 represents occipitallobe, putamen, substantia nigra, temporal lobe, thalamus, subthalmic nucleus, spinal chord, respectively; C 1-8 represents heart, aorta, skeletal muscle, colon, bladder, uterus, prostate, stomach, respectively; D 1-8 represents testis, ovary, pancreas, pituitary gland, adrenal gland, thyroid gland, salivary gland, mammary gland, respectively; E 1-8 represents kidney, liver, small intestine, spleen, thymus, peripheral leukocyte, lymph node, bone marrow, respectively; F 1-4 represents appendix, lung, trachea, placenta, respectively; G 1-7 represents (All Fetal) brain, heart, kidney, liver, spleen, thymus, lung, respectively; and H 1-8 represents 100 ng total yeast RNA, 100 ng yeast tRNA, 100 ng E. coli rRNA, 100 ng E. coli DNA, 100 ng poly r(A), 100 ng Cot 1 human DNA, 100 ng human DNA, 500 ng human DNA, respectively.

### DETAILED DESCRIPTION OF THE INVENTION

Almost 50 years have elapsed since the independent discoveries of capacitation by Chang and Austin, but molecular mechanisms to explain this process are not yet fully understood. Studies of CBP86 have now provided an added dimension to the understanding of capacitation related molecular events in the flagellum. The observations that a new calcium binding protein (CBP86) exists in the sperm tail throughout the entire length of the principle piece in association with the fibrous sheath adds another possible molecular component to the calcium signaling pathway active during hyperactivation. CBP86 may be involved in calcium sequestration and episodic release and thus may play a direct role in flagellar motility.

As reported herein the 86 kDa ⁴⁵Ca binding isoforms of CBP86 are composed of subunits. These isoforms increase during in vitro capacitation, and dephosphorylation abolishes both calcium binding capacity and assembly of the 86 kDa isoforms. These observations point to a role for capacitation dependent phosphorylation in calcium signaling. Although the time course for capacitation in vitro differs from species to species a median time for in vitro capacitation of human sperm is three hours. This time course is similar to that observed for CBP86 phosphorylation and assembly, leading to the hypothesis that oligomerization of CBP86 into its calcium binding form is a capacitation related event requiring tyrosine phosphorylation and that the time required for this process may underlie the temporal requirements for capacitation and hyperactivation.

Furthermore, Northern and dot blot analysis of an extensive panel of tissues place CBP86 in the category of a sperm and testis-specific protein. Immunohistochemical analysis of human testis indicated that the CBP86 gene first becomes translated following meiosis and that the protein is present only in spermatids, moving to the flagellum during the final stages of spermatogenesis. In contrast to calmodulin, which is considered to sequester sperm Ca²⁺ but is present in many somatic cell types, the tissue specificity of CBP86 may provide a unique opportunity to target calcium sequestration and signaling in sperm. In addition, the post-meiotic pattern of protein localization and the tissue specificity of gene expression indicate that CBP86 should be given consideration as a candidate for targeted male contraception because of the possibility that antagonists of CBP86 might act selectively during spermatogenesis.

Accordingly, the present invention is directed to therapeutic and diagnostic methods and compositions based on CBP86 proteins and nucleic acids. Antagonists of CBP86 function can be used to interfere with the capacitation of vertebrate sperm, and thus used as contraceptive agents. Furthermore, antibodies against the CBP86 protein can be used for the diagnosis of conditions or diseases characterized by expression or overexpression of CBP86, or in assays to monitor patients being treated with CBP86 agonists, antagonists or inhibitors.

In one embodiment, the present invention is directed to a purified polypeptide comprising the amino acid sequence of SEQ ID NO: 2, or an amino acid sequence that differs from SEQ ID NO: 2 by one or more conservative amino acid substitutions. More preferably, the purified polypeptide comprises an amino acid sequence that differs from SEQ ID NO: 2 by 20 or less conservative amino acid substitutions, and more preferably by 10 or less conservative amino acid substitutions. Alternatively, the polypeptide may comprise an amino acid sequence that differs from SEQ ID NO: 2 by 1 to 5 alterations, wherein the alterations are independently selected from a single amino acid deletion, insertion or substitution.

Another embodiment of the present invention encompasses truncated versions of the polypeptide of SEQ ID NO: 2, wherein the polypeptide is translated from one of several alternative start codons located downstream from the first start codon, at positions 343, 583, 631 and 652, respectively. For example, the polypeptide may comprise the sequence of SEQ ID NO: 2, SEQ ID NO: 3, SEQ ID NO: 4, SEQ ID NO: 5 or SEQ ID NO: 6, or an amino acid sequence that differs from SEQ ID NO: 2, SEQ ID NO: 3, SEQ ID NO: 4, SEQ ID NO: 5 or SEQ ID NO: 6 by one or more conservative amino acid substitutions, more preferably, by 10 or less conservative amino acid substitutions.

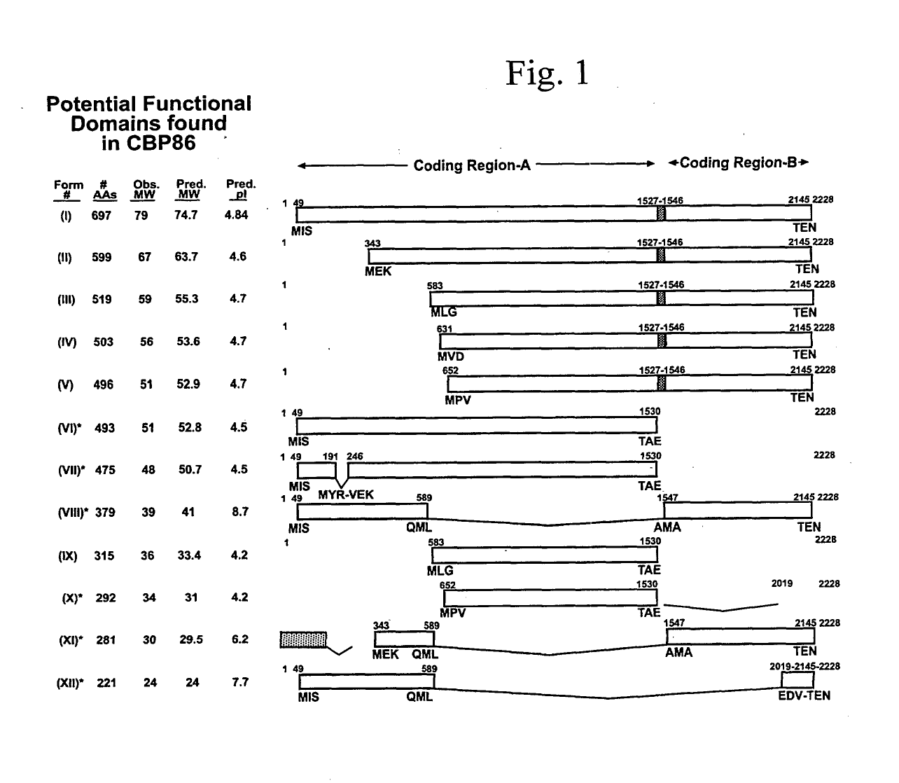

The present invention also comprises the various alternative spliced forms of the CBP86 proteins as shown in FIG. 1. In particular, the present invention is directed to a polypeptide comprising the sequence of SEQ ID NO: 15 or an amino acid sequence that differs from SEQ ID NO: 15 by one or more conservative amino acid substitutions. In another embodiment, the polypeptide comprises the sequence of SEQ ID NO: 16 or an amino acid sequence that differs from SEQ ID NO: 16 by one or more conservative amino acid substitutions.

The CBP86 proteins also contain a number of binding motifs. Three of the 6 known motifs of catapase, which are the signatures for the P-type ATPase cation transport superfamily are found in CR-A of CBP86:

| | |
|---|---|
| LKTLLEGISR | (SEQ ID NO: 7) |
| VSDNTGQEESGENSV | (SEQ ID NO: 8) |
| SGTSVKSSSGP | (SEQ ID NO: 9) |

The N-terminus of CR-A contains 3 of 4 possible motifs that constitute SH3 domains:

| | |
|---|---|
| NQFAAAYFQEL | (SEQ ID NO: 10) |
| VEKWSEGTTP | (SEQ ID NO: 11) |
| KTTQFPSVYAVPG | (SEQ ID NO: 12) |

Further computer analysis found two (5 and 6) of the possible eight progesterone receptor motifs:

| | |
|---|---|
| PSSPPPTAVSPEFAYVP | (SEQ ID NO: 13) |
| AEATALLSDTSLKGQPE | (SEQ ID NO: 14) |

In one embodiment, the present invention provides methods of screening for agents, small molecules, or proteins that interact with CBP86. The invention encompasses both in vivo and in vitro assays to screen small molecules, compounds, recombinant proteins, peptides, nucleic acids, antibodies etc. which bind to or modulate the activity of CBP86 and are thus useful as therapeutics or diagnostic markers for fertility.

In one embodiment the CBP86 polypeptide, or bioactive fragments thereof, is used to isolate ligands that bind to the CBP86 polypeptide under physiological conditions. The method comprises the steps of contacting the CBP86 polypeptide with a mixture of compounds under physiological conditions, removing unbound and non-specifically bound material, and isolating the compounds that remain bound to the CBP86 polypeptides. Typically, the CBP86 polypeptide will be bound to a solid support using standard techniques to allow rapid screening compounds. The solid support can be selected from any surface that has been used to immobilize biological compounds and includes but is not limited to polystyrene, agarose, silica or nitrocellulose. In one embodiment the solid surface comprises functionalized silica or agarose beads. Screening for such compounds can be accomplished using libraries of pharmaceutical agents and standard techniques known to the skilled practitioner.

The present invention also encompasses nucleic acid sequences that encode the CBP86 polypeptide, and bioactive fragments and derivatives thereof. In particular the present invention is directed to nucleic acid sequences comprising the sequence of SEQ ID NO: 1 or fragments thereof. In one embodiment, purified nucleic acids comprising at least 8 contiguous nucleotides (i.e., a hybridizable portion) that are identical to any 8 contiguous nucleotides of SEQ ID NO: 1 are provided. In other embodiments, the nucleic acids comprises at least 25 (contiguous) nucleotides, 50 nucleotides, 100 nucleotides, 200 nucleotides, or 500 nucleotides of SEQ ID NO: 1. In one embodiment the nucleic acid sequence comprises a 350 bp nucleic acid sequence that is identical to a contiguous 350 bp sequence of SEQ ID NO: 1. In another embodiment the nucleic acid sequence comprises the sequence of SEQ ID NO: 25 or SEQ ID NO: 26.

The present invention also includes nucleic acids that hybridize (under conditions defined herein) to all or a portion of the nucleotide sequence represented by SEQ ID NO: 1 or its complement. The hybridizing portion of the hybridizing nucleic acids is typically at least 15 (e.g., 20, 25, 30, or 50) nucleotides in length. Hybridizing nucleic acids of the type described herein can be used, for example, as a cloning probe, a primer (e.g., a PCR primer), or a diagnostic probe. It is anticipated that the DNA sequence of SEQ ID NO: 1, or fragments thereof can be used as probes to detect additional members of the CBP86 families and to detect homologous genes from other vertebrate species.

Nucleic acid duplex or hybrid stability is. expressed as the melting temperature or Tm, which is the temperature at which a nucleic acid duplex dissociates into its component single stranded DNAs. This melting temperature is used to define the required stringency conditions. Typically a 1% mismatch results in a 1° C. decrease in the Tm, and the temperature of the final wash in the hybridization reaction is reduced accordingly (for example, if two sequences having >95% identity, the final wash temperature is decreased from the Tm by 5° C). In practice, the change in Tm can be between 0.5° C. and 1.5° C. per 1% mismatch.

The present invention is directed to the nucleic acid sequence of SEQ ID NO: 1 and nucleic acid sequences that hybridize to that sequence (or fragments thereof) under stringent or highly stringent conditions. In accordance with the present invention highly stringent conditions are defined as conducting the hybridization and wash conditions at no lower than −5° C. Tm. Stringent conditions are defined as involve hybridizing at 68° C. in 5× SSC/5× Denhardt's solution/1.0% SDS, and washing in 0.2× SSC/0.1% SDS at 68° C . Moderately stringent conditions include hybridizing at 68° C. in 5× SSC/5× Denhardt's solution/1.0% SDS and washing in 3× SSC/0.1% SDS at 42° C. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2. 10.

In another embodiment of the present invention, nucleic acid sequences encoding the CBP86 receptor can be inserted into expression vectors and used to transfect cells to enhance the expression of those receptors on the target cells. In accordance with one embodiment, nucleic acid sequences encoding CBP86, or a fragment or a derivative thereof, are inserted into a eukaryotic expression vector in a manner that operably links the gene sequences to the appropriate regulatory sequences, and CBP86 is expressed in a eukaryotic host cell. Suitable eukaryotic host cells and vectors are known to those skilled in the art. In particular, nucleic acid sequences encoding CBP86 may be added to a cell or cells in vitro or in vivo using delivery mechanisms such as liposomes, viral based vectors, or microinjection. Accordingly, one aspect of the present invention is directed to transgenic cell lines that contain recombinant genes that express CBP86.

Another embodiment of the present invention comprises antibodies that are generated against CBP86. These antibodies can be formulated with standard carriers and optionally labeled to prepare therapeutic or diagnostic compositions. Antibodies to CBP86 may be generated using methods that are well known in the art. Such antibodies may include, but are not limited to, polyclonal, monoclonal, chimeric (i.e. “humanized” antibodies), single chain (recombinant), Fab fragments, and fragments produced by a Fab expression library. These antibodies can be used as diagnostic agents for the diagnosis of conditions or diseases characterized by expression or overexpression of CBP86, or in assays to monitor patients being treated with CBP86 receptor agonists, antagonists or inhibitors. The antibodies useful for diagnostic purposes may be prepared in the same manner as those described above for therapeutics. The antibodies may be used with or without modification, and may be labeled by joining them, either covalently or non-covalently, with a reporter molecule.

In accordance with one embodiment an antibody is provided that specifically binds to the protein of SEQ ID NO: 2. More particularly, the antibody binds to the amino acid sequence of SEQ ID NO: 15. Alternatively, the antibody specifically binds to the amino acid sequence of SEQ ID NO: 16. In one preferred embodiment the antibody is a monoclonal antibody.

The invention also encompasses antibodies, including anti-idiotypic antibodies, antagonists and agonists, as well as compounds or nucleotide constructs that inhibit expression of the CBP86 gene (transcription factor inhibitors, antisense and ribozyme molecules, or gene or regulatory sequence replacement constructs), or promote expression of CBP86 (e.g., expression constructs in which CBP86 coding sequences are operatively associated with expression control elements such as promoters, promoter/enhancers, etc.).

The present invention also encompasses compositions that can be placed in contact with sperm cells to inhibit the function of the CBP86 protein (i.e. either by inhibiting the expression of the CBP86 protein or by interfering with the protein's function). In particular the compositions may comprise peptide fragments of CBP86, or analogs thereof that are taken up by the sperm cells and compete for binding with CBP86's natural ligands. Such inhibitory peptides can be modified to include fatty acid side chains to assist the peptides in penetrating the sperm cell membrane. Compositions comprising a CBP86 inhibitory agent can be used to modulate fertility of an individual, and in one embodiment, the inhibitory agents function as a male contraceptive pharmaceutical. In accordance with one embodiment a composition is provided that comprises an eight to fifteen amino acid sequence that is identical to an eight to fifteen consecutive amino acid sequence of SEQ ID NO: 2 and a pharmaceutically acceptable carrier.

The CBP86 protein contains a number of protein binding domains, including three SH3 domains located at the 5′ end of CR-A of the CBP86 gene. In addition, the 3′ end of CR-A and the 5′ end of the CR-B are relatively proline rich. Both the SH3 domains and the proline-rich stretches, referred to herein as the putative dimerization domains, provide CBP86 with potential sites for interaction with other flagellar proteins (such as the AKAPs). In accordance with one embodiment of the present invention the CBP86 polypeptide is used in an assay to screen for compounds that interfere with CBP's ability to bind to AKAPS. The assay comprises combining CRP with an AKAP in the presence of one or more potential inhibitors to monitor the ability of the potential inhibitor to prevent AKAP binding to the CBP polypeptide and/or the ability of the potential inhibitor to disrupt AKAP/CBP complexes. Inhibitor of such binding interactions have utility as contraceptive agents due to their ability to prevent capacitation of sperm cells.

The CBP86 polypeptide and its splice derivatives can also be used in accordance with the present invention as a marker for determining the extent of capacitation of sperm cells present in a sperm sample. The assay is based on the premise that phosphorylation and formation of the 86 kDa isoform of CBP86 is correlated with capacitation of the sperm cells. Therefore measuring the phosphorylation or oligomerization of CBP86 serves as a marker of capacitation.

### EXAMPLE 1

### Isolation of the CBP86 Protein

Materials and Methods

Solubilization and Electrophoresis of Human Spermatozoal Proteins

Preparation of semen specimens and solubilization of sperm proteins were performed as previously described (Naaby-Hansen et al, 1997a.) For analytical two-dimensional electrophoresis the detergent/urea extracted proteins were separated by isoelectric focusing (IEF) in acrylamide tube gels prior to second dimensional gel electrophoresis (SDS-PAGE), which was performed in a Protean II xi Multi-Cell apparatus (Bio-Rad, Richmond, Calif.) or on large format (23×23 cm) gels (Investigator 2-D Electrophoresis System, ESA) which were also employed for preparative 2D gel electrophoresis. Electrotransfer to nitrocellulose membranes and subsequent visualizing of the proteins by gold staining was accomplished as previously described (Naaby-Hansen et al, 1997) while electrotransfer to PVDF membranes (0.2 mm pore size, Pierce) was carried out as described by Henzel et al. (1993) using the transfer buffer composition of Matsudaira (1987) (10 mM 3-[cyclohexylamino]-1-propanesulfonic acid, 10% methanol, pH 11). The immobilized proteins were visualized by staining in a solution containing 0.1% Commassie R250, 40% methanol and 0.1% acetic acid for one minute, followed by destaining in a solution of 10% acetic acid and 50% methanol for 3×3 minutes.

In Vitro Capacitation

Motile sperm were harvested by the swim up method of Bronson and Fusi (1990). A control sample was removed and snap frozen (−70 C.), while the remaining sperm were resuspended in one of the following media: Dulbecco's PBS, BWW, BWW plus 3 mM b-cyclodextran (Sigma), BWW plus b-cyclodextran and 100 μμM progesterone, human tubal fluid [HTF] (Irvine) plus HSA (30 mg/ml), HTF plus HSA plus 2, 20 or 100 μμM progesterone, HTF plus HSA plus 100 μμM progesterone plus either 100, 200 or 400 μμM of genestein or daidzein. (Akiyama et al., 1987). Capacitation was achieved by incubating the samples at 37° C. in 5% CO₂ with sperm removed at various timepoints and isolated by centrifugation.

Detection of Calcium Binding Proteins

Calcium binding proteins were demonstrated using a ⁴⁵Ca overlay assay modified from that described by Maruyama et al. (1984). The experiment was replicated 4 times. In brief, the 2-D gel separated proteins were transferred to PVDF membranes (Jethmalani et al., 1994), and the membranes were washed 3×20 min in a washing buffer (10 mM imidazole HCl, 60 mM KCl and 5 mM MgCl₂, pH 6.8) and incubated with 2 mCi/ml of ⁴⁵CaCl₂ in washing buffer for 30 min at room temperature. The membranes were subsequently rinsed for 2 min in distilled H₂O followed by 30 sec rinsing in 50% ethanol and were air dried on filterpaper for 15-20 min. The membranes were then dried by hot air from a hairdryer and exposed on phospho-imaging screens (Molecular Dynamics) for 10 days. The use of PVDF, shortening of the final wash steps, and employment of phospho-imaging detection increased the signal to noise ratio compared to that achieved with the procedure originally proposed by Maruyama et al (1984). Some of the PVDF membranes were subsequently stained with Commassie to localize the calcium binding proteins within the total 2-D protein pattern, while other membranes were used for western blot analysis as described below. Computerized pattern analysis and densitometry of the autoradiograms and the stained membranes were performed employing 2D Analyzer software (BioImage 2000).

Generation of Antiserum Against Gel Purified CBP86

The 86 kDa Coomassie-stained protein spot was cored from three 1.5 mm thick 2-D SDS-PAGE gels of human sperm extracts. The gel cylinders were minced into a slurry in 1 ml of PBS and emulsified with an equal volume of complete Freunds adjuvant. Six hundred ul of this emulsion was intradermally injected into a New Zealand white rabbit, followed by two monthly subcutaneous booster injections of similarly-prepared antigen with incomplete Freunds adjuvant. Serum was collected 10 days after each booster injection.

Dephosphorylation of Sperm Proteins

To examine the relationship between phosphorylation and calcium binding capacity of the 86 kDa CBP86 form, sperm from 4 individuals were capacitated for 5 hr in HTF plus albumin, and the sperm were extracted in NP40/urea and the extracts pooled. The lysate was divided and one aliquot was treated with 2 U/ml calf intestinal alkaline phosphatase (Boehringer Manheim) for ½ hour at 37° C. while the other aliquot remained untreated.

Microsequencing of the 86 kDa Calcium Binding Tyrosine Phosphorylated Protein

The 86 kDa Coomassie stained protein spot was cored from a 1.5 mm thick 2D SDS-polyacrylamide gel and fragmented into smaller pieces. The protein was destained in methanol, reduced in 10 mM dithiothreitol and alkylated in 50 mM iodoacetamide in 0.1 M ammonium bicarbonate. After removing the reagents, the gel pieces were incubated with 12.5 ng/ml trypsin in 50 mM ammonium bicarbonate overnight at 37° C. Peptides were extracted from the gel pieces in 50% acetonitrile in 5% formic acid and microsequenced by tandem mass spectrometry and by Edman degradation at the Biomolecular Research Facility of the University of Virginia. Five peptide sequences were obtained by mass spectrometry:

| | |
|---|---|
| LVVPYGLK | (SEQ ID NO: 17) |
| TLLEGISR | (SEQ ID NO: 18) |
| TNPSNINQFAAAYFQELTMYR | (SEQ ID NO: 19) |
| KYSSVYMEAEATALLSDTSL | (SEQ ID NO: 20) |
| GQPEVPAQLLDAEGAI | (SEQ ID NO: 21) |

Differentiation of leucine and isoleucine in the sequences were determined by Edman sequencing of HPLC isolated peptides.

Cloning, Sequencing and Analysis of cDNAs

A degenerate deoxyinosine containing sense primer (5′-GGI-CAG-CCI-GAG-GTI-CCI-GCI-CAA/G-C/TT-3′) (SEQ ID NO: 22) was designed from peptide number 5 (GQPEVPAQL; SEQ ID NO: 23) and obtained from GIBCO BRL (Life Technologies, CA). Using this forward primer and an adapter primer (AP1), a 3′-RACE (rapid amplification of cDNA ends) PCR was performed with 0.25 ng of human testicular Marathon ready cDNA (CLONTECH, CA). in a 25 μl assay system for 40 cycles. Thermal cycling was done in a MJ Research (Watertown, Mass). thermal cycler (PTC-200 DNA engine) using a program of one 3 min. cycle at 94° C. followed by 40 cycles of denaturation, annealing and elongation at 94° C. for 30 sec, 60° C. for 1 min and 68° C. for 2 min. PCR products were separated on a 1.7% NuSieve (FMC, ME) agarose gel and a unique 1.0 kb DNA fragment was reamplified, cloned into the pCR 2.1-TOPO vector (Invitrogen, CA), and sequenced on a Perkin-Elmer Applied Biosystems DNA sequencer using BigDyeÔ fluoresence dye terminator chemistry with Taq DNA polymerase (Perkin-Elmer, NJ). The 3′ clone contained 1001 bp including a portion of CR-A and all of CR-B. The 5′ end of the cDNA was also amplified by 5′ RACE PCR from the same template using an adapter primer (AP1) and an antisense 3′ gene-specific primer (5′-TTA-TTC-AGC-TGT-TGA-TTC-CCC-TTC-TGG-TTC-AAT-TTC-TGG -3′) (SEQ ID NO: 24) which was 263 bp downstream from the 5′ end of the 1.0 kb 3′ clone. A product of 1530 bp was obtained and cloned into the pCR 2.1-TOPO vector. The 5′ clone revealed a 48 bp untranslated region and an open reading frame of 1479 bp. The cDNA clones were sequenced in both directions using vector-derived and insert-specific primers. The nucleotide and amino acid sequence data were assembled.

Cloning of alternatively spliced forms of the transcript was performed by probing a 5′-Stretch λλDR2 human testis cDNA library (Clontech, CA) according to manufacturers instructions with the full-length ³²P-labeled cDNA obtained through the RACE protocol. Purified tertiary plaques were converted to their plasmid forms, plated, grown in LB broth and the plasmid DNA isolated by Qiagen Mini-Kit columns before sequencing with both plasmid and gene-specific primers.

Northern and Dot Blot Analyses

A Northern blot containing 2 mg of poly(A)⁺ RNA from eight selected human tissues and a normalized RNA dot blot containing 89 to 514 ng of mRNA from 50 different human tissues were obtained from Clontech. The Northern blot was probed with a ³²P-labeled 1479 bp DNA corresponding to bp 49-1527 of CR-A. Probes were prepared by random oligonucleotide prime labeling (Feinberg and Vogelstein, 1983). Hybridization was performed in ExpressHyb solution (Clontech) at 68° C. for 1 h followed by three washes in 2× SSC, 0.05% SDS at room temperature and two washes in 0.1× SSC, 0.1% SDS for 20 min at 50° C. The blot was exposed to X-ray film at −70° C. for 60 h with two intensifying screens. The dot blot was probed with the same ³²P-labeled cDNA corresponding to coding region A. The blot was hybridized in ExpressHyb solution (Clontech) containing salmon sperm DNA and human placental Cot-1 DNA overnight at 65° C. The blot was then washed three times in 2× SSC, 1% SDS at 65° C. followed by two additional washes in 0.1× SSC, 0.5% SDS at 55° C. before exposing the filter to X-Ray film for 18 h at −70° C. with two intensifying screens.

Reduction and Carboxymethylation of Human Sperm Proteins.

Washed sperm samples (Naaby-Hansen et al, 1997) were extracted in 8 M urea in 0.36 M Tris-HCl, pH 8.6 containing 2% NP40 for 1 h at 4° C. The supernatant was precipitated, washed twice in 80% ethanol (final) and reconstituted in the urea buffer with no NP40. An aliquot of 1.5 mg protein was incubated in 8 M urea, 0.2% EDTA, 119 mM mercaptoethanol in 0.36 M Tris-HCl, pH 8.6 at room temp for 4 h under nitrogen in screw-cap tubes (Crestfield et al, 1963). The mix was then treated with a freshly prepared solution of iodoacetic acid (0.111 M final concentration) in 1 N NaOH for 15 min at room temp in the dark. After the reaction, the carboxymethylated proteins were washed in ethanol and used for Western analyses.

Expression and Purification of the Recombinant Protein and Antibody Production

The cDNA encoding CR-A of CBP86 was amplified by polymerase chain reaction from human testicular Marathon ready cDNA (Clontech). Primers were designed to create a NcoI site at the 5′ end and a Not I site at the 3′ end of the polymerase chain reaction product. The amplified cDNA was cloned into the NcoI-Not I sites of the pET28b expression vector (Novagen) and Escherichia coli strain NovaBlue(DE3) was transformed with the plasmid construct. The resulting construct appended six residues of histidine tag on the C-terminus of the protein. The expression plasmid construct was sequenced at the 5′ and 3′ ends to verify the reading frame of the construct.

A single positive colony was inoculated in 1 liter of LB broth with 30 mg/ml kanamycin and grown at 37° C. until the A₆₀₀ reached 0.6. Then recombinant protein expression was induced by addition of 1.0 mM IPTG (isopropyl-1-thio-b-D-galactopyranoside), and growth was continued for another 3.0 h. The cells were pelleted, resuspended in 1× binding buffer (20 mM Tris-HCl, pH 7.9, 0.5 M NaCl, 5 mM imidazole) containing 0.1% NP40 (Sigma) and 0.1 mg/ml lysozyme on ice for 30 min, and sonicated briefly. The insoluble pellet resulting from centrifugation at 15000 × g for 15 min was dissolved in 6 M urea in 1× binding buffer for 1 h on ice. After recentrifugation at 15000 × g for 15 min the urea soluble fraction was loaded onto a Ni^{2 +}-activated His-Binding resin column (Novagen) following manufacturers protocol, and the recombinant protein was eluted with 300 mM immidazole in 1× binding buffer containing 6 M urea. The affinity purified recombinant protein was used for immunization of female Lewis rats (200 ug/rat) in Freunds complete adjuvant. Animals were boosted twice at an interval of 14 days with 200 μg of recombinant protein in incomplete Freunds adjuvant and serum was collected 7 days after each boost.

Immuno-blotting

Western blotting was performed employing a 1:3500 dilution of the rabbit antiserum raised against gel purified CBP86 antigen and a 1:2500 dilution of the rat antiserum to rCBP86. Sperm proteins phosphorylated on tyrosine residues were identified by immunoblotting with horseradish peroxidase-conjugated anti-phosphotyrosine monoclonal antibody RC-20 (Transduction Laboratories) at a 1:2500 dilution in 10 mM Tris (pH 7.5), 0.1 M NaCl, and 0.05% Tween 20 for 20 min at 37° C. (Ruff-Jamisson et al, 1993)

Diagonal Gels

Human sperm cells, purified by swim-up, were solubilized for 20 min at 22° C. in Laemmli sample buffer (600×10⁶ cells/ml), lacking beta-mercaptoethanol and containing 2 mM PMSF and 5 mM EDTA to inhibit protease activity. The supernatant was heated and 50 l/lane were loaded on SDS-PAGE gradient gels (5-12%) with a 5% stacking gel. Afterwards the gel was cut into strips (lanes) and some strips were incubated for 45 min at 37° C. in reducing buffer (0.5% (w/v) DTT, 0.1% (w/v) SDS, 125 mM Tris, pH 6.8). Reduced and unreduced Gel-strips were then laid horizontally on top of 7.5% SDS-PAGE gels and proteins were run out. Proteins were transferred to nitrocellulose membranes and probed with anti-rec-CBP86 as above.

Localization of CBP86 in the Seminiferous Epithelium of Human Testis

Testes were obtained from three patients undergoing elective orchiectomies. Testes were sliced once with a razor blade and immersed in neutral buffered formalin (4%) solution (Sigma) for one hour. The tissue was then minced and placed into fresh fixative overnight. The tissue was dehydrated in a graded series of ethanols, cleared in xylene, and embedded in paraffin. 2.5 μm thick sections were cut, mounted onto slides, de-paraffinized, rehydrated and permeabilized with 100% methanol. Sections were incubated in blocking solution containing 10% NGS in PBS, incubated with anti-rCBP86 antiserum or pre-immune serum (1:200) in PBS containing 1% NGS (PBS-NGS), washed, incubated with FITC-labeled goat anti-rat IgG (1:400; Jackson Immunoresearch) in PBS-NGS, washed, and mounted with Slow Fade (Molecular Probes, Eugene, Oreg.) containing DAPI II counterstain (Vysis, Downers Grove, Ill.). Sections were observed by epifluorescence microscopy using a Zeiss microscope. Individual blue and green fluorescent images were obtained using a digital camera (Hamamatsu) and compiled using Openlab software (Improvision Inc., Boston, Mass).

Indirect Immunofluorescence of Human Sperm

For immunofluorescence studies fresh human sperm were harvested over a discontinuous 55%/80% Percoll gradient and subsequently washed 3× with Hams F-10 media. The sperm were counted using a hemocytometer and diluted to a concentration of 1×10⁶ sperm/ml. A 20 μl aliquot of the sperm suspension was added per well (2×10⁵ sperm) onto poly-L-lysine coated slides. The slides were dried at 40° C. and then methanol fixed for 10 min. In some experiments no fixation was performed and the sperm were simply air dried onto the slide. After washing 3×5 min in PBS, the slides were frozen at −70° C. for 1 week. All subsequent incubations were done in a humid chamber. The preparations were blocked in 10% normal goat serum (NGS) in PBS with 0.05% Tween-20 (PBS-tw) for 30 min. The primary antiserum, either rabbit anti CBP86 antiserum or rat anti recombinant CBP86 and their pre-immune controls, was diluted 400-fold with 10% NGS in PBS-tw and were incubated with the specimen overnight at 4° C. The slides were then washed 3×5 min in PBS-tw, and the secondary antibody, goat anti-rabbit IgG FITC conjugated (Jackson ImmunoResearch) or goat anti-rat IgG FITC conjugated (Jackson ImmunoResearch), were applied at 1:200 dilutions in 10% NGS in PBS-tw for 1 hour at 37° C. The slides were washed 3×5 min in PBS-tw, and Slow Fade-Light Antifade Kit (Molecular Probes, Inc.) was used to reduce the fading rate of the fluorescein.

Electron Microscopic Localization

Sperm from four donors were pooled and washed twice by centrifugation at 550 × g in wash buffer, (Ham's F10 Nutrient Mixture (Gibco/BRL) with 3% sucrose). The washed sperm were resuspended in fixative consisting of 4% paraformaldehyde and 0.2% glutaraldehyde in wash buffer for 15 minutes at room temperature. After removing fixative by centrifugation and washing 3× with wash buffer, the sperm were dehydrated through a graded series of ethanols from 40% to 100%. The cells were infiltrated with and embedded in Lowicryl K4M (Electron Microscopy Sciences, Ft. Washington, Pa.) according to the manufacturer's recommendations. The blocks were polymerized with UV light for 72 hrs at −20° C. and ultrathin sections of 100 nm thickness were cut.

Non-specific sperm-antibody interactions were blocked by incubating the sections in undiluted normal goat serum for 15 minutes at room temperature and washing once with wash buffer. Rat antiserum to rCBP86 and pre-immune serum were diluted 1:50 in wash buffer with 1% normal goat serum, 1% bovine serum albumin and 0.05% Tween 20. Lowicryl sections were incubated with diluted anti-rCBP86 or wash buffer alone at 4° C. for 16 hours. After washing four times in wash buffer, they were incubated for 1.5 hours at room temperature with 5 nm gold-conjugated secondary antibody, goat anti-rat IgG (Goldmark Biologicals, Phillipsburg N.J.) diluted 1:35 in wash buffer. The sections were washed with distilled water and stained with uranyl acetate before examination with a JEOL 100CX electron microscope.

In Vitro Phosphorylation of Recombinant CBP86 with c-Src

Baculovirus expressed c-Src was purchased from Upstate Biotechnology, Inc. (Lake Placid, N.Y). Recombinant CBP86 was phosphorylated by c-Src in an in vitro kinase assay in which 0, 0.8, 0.16, or 0.03 μμg of CBP86 was incubated in the presence or absence of 1 unit of c-Src in a 50 μμl reaction containing 50 mM HEPES, pH 7.4, 5 mM MnCl₂, 70 nM ATP, 10 Ci [³²P]ATP (6000 Ci/mmol) for 10 min. The reaction was terminated with Laemlli SDS sample buffer and subjected to SDS-PAGE and autoradiography.

Results

Identification and Characterization of Calcium Binding Proteins (CBPs) in Human Spermatozoa.

The ⁴⁵Ca overlay technique of Maruyama et al (1983) was employed on 2-D blots of human sperm proteins to identify more than 20 calcium binding protein spots (CBPs) in the range of 12.5 kDa to 115 kDa and pIs of 3.8 to 5.3. The relative intensity of each spot, indicative of the concentration of the binding protein and/or its calcium binding capacity, was determined by computer densitometry. More than 90% of the ⁴⁵Ca was bound by eleven major CBPs migrating at MWs (kDa)/pI of 86/4.0, 80.4/4.3, 60.5/4.2, 55/4.9, 55/5.25, 26.5/5.2, 25/4.6, 24.7/4.75, 16.5/3.9, 15.8/4.7 and 14.5/3.95 in four replications of the experiment. The ⁴⁵Ca overlay procedure, which was conducted at pH 6.8, did not detect human sperm CBP's in the neutral and basic areas (pH 6.2-8.5) of the IEF/PAGE gels. The protein which bound the majority (60%) of the ⁴⁵Ca was identified as calmodulin (CaM) based on its electrophoretic migration at 16.5 kDa and pI of 3.9.

Three prominent calcium binding proteins migrating at 86 (84-88) kDa/4.0 (3.9-4.1), 60.5 kDa/4.2 and 26.5 kDa/5.2 were excised and microsequenced by CAD Mass Spectrometry (MS). Five internal peptide sequences and 15 N-terminal amino acids were obtained from the 60.5 kDa CBP, which identified the protein as calreticulin (CRT). A 26.5 kDa CBP was previously identified as a human sperm surface protein by vectorial labeling with ¹²⁵I and was also detected in human seminal fluid. Six peptide sequences obtained by MS and 22 N-terminal amino acids obtained by Edman degradation identified the 26.5 kDa CBP as serum amyloid P-component precursor (SAP). Calcium binding to CaM and SAP resides within EF-hand motifs, while CRT's calcium binding occurs in repeated, polyacidic C-terminal domains. The ability of these proteins to bind ⁴⁵Ca validated the sensitivity and specificity of the ⁴⁵Ca overlay procedure on 2-D gels.

The 86 kDa region of the gel which contained a train of protein spots which readily bound ⁴⁵Ca, was designated calcium binding protein 86 [CBP86]. Densitometry of the ⁴⁵Ca overlays indicated CBP86 was the second most intense staining region on the 2-D image after calmodulin. MS microsequence data from 5 peptides obtained after tryptic digestion of the excised 86 kDa spot (SEQ ID NOS: 17-21) did not match any known peptide sequences in any protein or gene database. Silver staining showed several isoforms of CBP86 varying slightly in mass and charge. The acidic isoforms of CBP86 bound more calcium than the more basic isoforms even though the two differentially charged groups of CBP86 showed similar staining with silver nitrate. The acidic CBP86 isoforms appeared to be more readily soluble than the basic isoforms because they appeared after only 20 seconds of solubilization in non-ionic detergent/urea when little if any of the basic 86 kDa isoforms were solubilized.

CBP86 Variants Showed Shifts in PI after Dephosphorylation.

The central, dense portion of the 86 kDa protein cluster was excised from several preparative 2-D gels and a rabbit antiserum was raised to the gel purified proteins. On 2-D immunoblots this antiserum recognized the 86 kDa immunogen (as well as prominent clusters of protein spots at 27-38, 38-42, 50-56, and 63-72, each of which showed charge heterogeneity. Western blots of sperm proteins that had been solubilized in the presence of calf intestinal alkaline phosphatase resulted in the virtual disappearance of the more acidic 86 kDa immunoreactive isoforms although the more basic isoforms remained. In addition, isoforms in the 38-42 and 50-56 kDa clusters shifted to more basic pIs after phosphatase treatment, indicating that the charge heterogeneity of these CBP86 forms is in part due to phosphorylation.

Cloning of CBP86 and its Alternatively Spliced Variants

A degenerate inosine-containing forward primer designed from peptide number 5, GQPEVPAQL (SEQ ID NO: 23), was employed to amplify a 1.0 kb region of cDNA by 3′-RACE PCR from human testicular Marathon-Ready cDNA (Clontech, CA). A 1530 bp 5′-cDNA fragment, including a 48 bp untranslated region, was similarly amplified and cloned using standard 5′-RACE PCR with an antisense 3′ reverse primer generated to a sequence 263 bp downstream from the 5′-end of the 1.0 kb 3′-clone. A nucleotide sequence for a composite 2228 bp CBP86 cDNA (SEQ ID NO: 1) was obtained by sequencing the two PCR fragments in both directions. This 2228 bp cDNA was the longest CBP86 cDNA obtained. This cDNA was P³² labeled and employed to screen a human testicular λλDR2 5′-Stretch cDNA library (Clontech, CA). Phage isolates were digested with restriction endonuclease, grouped according to restriction fragment sizes, and sequenced to yield several cDNAs also of 2228 bp as well as five alternative splice variants, which were submitted to Genbank under accession numbers AF295037, AF29038, AF295039, AF329634 and AF007205.

The five splice variants and the 2228 bp CBP86 cDNA are noted by asterisk in FIG. 1 (forms VI-VII and X-XII). Analysis of these sequences led to the initial conclusion that the CBP86 sequence was divided into two coding regions, CR-A and CR-B. CR-A begins at bp 49 and ends at bp 1527 (codons 1-494) with a stop codon TAA at bp 1528-30 serving as an authentic termination codon for CR-A. CR-A encodes a predicted protein of 493 amino acids with a mass of 52.8 kDa and pI of 4.5. Eighteen in frame nucleotides [1531-1546] then separate CR-A from the ATG start codon [1547-1550] of CR-B. CR-B [nucleotides 1547-2145] encodes a peptide that serves as the carboxy terminus on several CBP86 variants. Splice variants were sequenced containing alternative start codons at bps 49-51 [clones VI, VII, VII and XII], bps 343-345 [clone XI], bps 583-585 [clone IX] or bps 652-654 [clone X]. Assuming the stop codon at bp 1528-30 was functional, these splice variants contained deletions of all of CR-B [clones VI, VII], a small N-terminal region of CR-A [clone VII], major portion of CR-A [clones VIII and XI], and a large domain spanning CR-A and B [clone XII].

To determine if the splice variants resulted in translated products human sperm proteins were reduced and carboxymethylated, separated on 1-D gels, and western blotted with an antisera raised to recombinant CR-A. Twelve immunoreactive CBP86 peptides ranging in apparent mass from 79 to 24 were identified. Isoforms at 67, 59 and 51 kDa were most immunoreactive. Importantly, CBP86 proteins were detected with masses higher than those predicted from coding region A or from any of the variants, including deletions of coding regions A or B or variants with splice junctions into coding region B. This observation, coupled to the fact that the intervening nucleotides between CR-A and CR-B were in-frame, led to the conclusion that a translational readthrough of the UAA translation terminating signal at the end of CR-A occurs in some instances. This translation readthrough accounts for the 12 translated peptides observed in vivo from the six variant cDNAs.

CBP86 Transcripts are Testis Specific

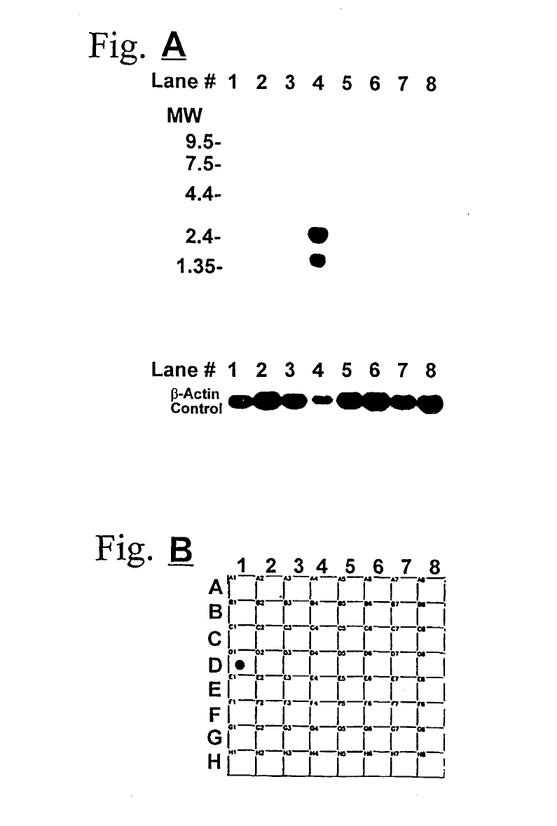

A ³²P-labeled cDNA probe corresponding to CR-A was employed for Northern analysis of mRNA from several tissues (FIG. 2A) and a dot blot (FIG. 2B) containing mRNAs from 50 distinct human tissues (Clontech, CA). Interestingly, two broad bands of approximately 2.4 and 1.4 Kb were noted in the testicular mRNA (FIG. 2A, lane 4), indicating that several CBP86 messages of different sizes were expressed in the human testis, a finding in concert with the cloning and sequencing of six cDNAs, including five splice variants noted above. The 2.4 kb transcript (FIG. 2A) detected in pooled human testicular mRNAs may be accounted for by the splice variants of forms I-VI and IX (cDNAs of 2228 bp) or form VII (2173 bp) assuming approximately 200 bp of untranslated region, while the 1.4 kb transcript may be accounted for by forms VIII (1270 bp) or X (1088 bp). A mRNA of approximately 0.9 to 1.0 kb is predicted for clone XII. Only a faint message of this size was detected on overexposed Northern blots, indicating that clone XII mRNAs as well as the 24 kDa protein are present in relatively lower abundance than other CBP86 mRNAs and proteins. Importantly, CBP86 transcripts were expressed in testis (FIG. 2A, lane 4 and FIG. 2B, spot D1) but not in other human tissues.

Motif Analysis of Splice Variants Revealed MAP4, RII Dimerization, and Extensin Domains

Analysis of the amino acid sequences deduced from the six CBP86 variants sequenced to date, assuming translation readthrough of the stop codon terminating CR-A, yields 12 predicted proteins ranging in mass from 24 to 74.7 kDa. (FIG. 1). Two of the predicted proteins [forms V and VI, FIG. 4] are nearly identical in mass (52.8 and 52.9 kDa). The masses for the 12 deduced proteins are several kDa less than the masses observed for the 12 reduced and blocked CBP86 translated proteins, indicating some post-translational modification(s) are occurring. Assuming several kDa of mass due to post-translational modification, the number and the pattern of the apparent masses of CBP86 proteins detected in reduced and carboxymethylated sperm protein extracts corresponds to both the number and the masses of the proteins predicted from the six variants.

All five of the tryptic peptides microsequenced by MS from the original 86 kDa spot excised from the 2-D gel were recovered in the predicted amino acid sequence of CR-A. This finding validated that cDNAs corresponding to the 86 kDa protein spot originally identified as a Ca^{2 +} binding protein and cored from preparative 2D gels had been cloned.

Computer analyses to ascertain functional domains of CBP86 revealed that amino acids 94-493 bore a 25% identity with amino acids 308-717 of human microtubule associated protein 4 (MAP4). However, the homologous region did not involve the microtubule binding domain of MAP4, nor were the 18-mer repeats characteristic of the microtubule binding domain of MAPs present in CR-A or B. A 98 amino acid stretch at the N-terminus of CBP86 (residues 10 to 108) bore 30% identity to the testis-specific sperm protein SP17. Importantly, embedded within this domain, sequence similarity to the regulatory subunit of type II cAMP-dependent protein kinase was noted. In particular, Val¹⁰-Leu⁴⁴ bore a 40% identity and 57% similarity to amino acids 7-41 of RIIαα (Newlon et al, 1999).

This amino terminal region of RII contains both the RII dimerization domain and the AKAP binding domain. This region also includes one domain with similarity to catatpase and one SH3 motif. Three of the 6 known motifs of catapase, which are the signatures for the P-type ATPase cation transport superfamily, were noted in CR-A of CBP86. A sub-family of this superfamily are Ca⁺²-pump ATPases which, like CBP86, have Ca⁺²-binding activity.

Motif analysis was employed to screen a list of proteins with weak overall homology to CBP86 for those proteins having a known interaction with calcium. Analysed in this way, the C-terminal third of CR-A revealed similarities with cation transporters in overlapping but distinct segments (e.g. a 98 residue region, Gln³⁶⁷-Gly,⁴⁶³ showed 25% identity and 45% conserved homology with the beta-3 regulatory subunit from the L-type voltage dependent calcium channel [Fugu rubripes]; while a 67 residue region, Ser⁴²⁸-Glu,⁴⁹³ revealed 34% identity and 42% conserved homology with the Na-Ca+K exchanger [Bos taurus]; and a 57 residue region, Glu³³¹-Leu³⁸⁷ revealed 19% identity and 50% conserved homology to the central domain of the ion-channel forming colocin 1A toxin [E. coli].

The N-terminus of CR-A contained 3 of 4 possible motifs that constitute SH3 domains. Such Src homology-3 domains serve as sites for intermolecular protein binding, interacting with proline-rich sequences on a range of signalling and cytoskeletal proteins. Three PXXP consensus motifs, the cognate sites for SH3 interaction, are present in CR-A [aa 396-399, 471-474, and 473-476) and three were present in CR-B (aa 211-214, 214-217, 326-329). No extended helical domains or transmembrane domains were apparent within CR-A or B. However, in view of the fact that CBP86 undergoes oligomerization (see below), it is noteworthy that four elements, each 22 amino acids in length, with similarity to the 7 element fingerprint for G-protein-coupled receptors were noted in CR-A at positions 185-206, 204-225, 295-316, and 455-476. Oligomerization of CBP86 may confer function on these elements.

Six potential phosphorylation sites for PKC, two phosphorylation sites for CKII as well as four tyrosine residues were present in the C-terminus of the CBP86 CR-A, suggesting that this region may be regulated by phosphorylation. Interestingly, this C-terminal domain, including two catatpase sites, was deleted in clones VIII, XI and XII suggesting that full length CBP86 differs in function from these splice variants.

Further computer analysis found two (5 and 6) of the possible eight progesterone receptor motifs. A region covering residues 17 to 102 shared a 20% identity with helix domains 9, 10, 11 and 12 of the progesterone receptor binding domain. 63% of the residues in this region were either identical or conservative replacements for the progesterone receptor binding domain. Potential N-linked glycosylation sites (residues 50, 109 and 237) and two potential O- glycosylation sites (residues 258-261; 467-468) were also detected within CR-A of the CBP86 sequence. The 5′ region of CR-B is proline rich and contains two proline triplets, while overall, CR-B contains three cysteine residues.

A BLAST search revealed the highest alignment score to be a 40% similarity (25% identity) between aa 225-329 of ORF-B and the proline-rich extensin glycoprotein found in plant cell walls (Keller and Lamb, 1989). Extensins are members of the hydroxyproline-rich glycoprotein family (HRGPs) and contain a characteristic pentapeptide repeat Ser-Pro₄ (Chen and Varner, 1985) which in CR-B may be represented by a modified Ser-Pro₃ domain at aa 212-215. Interestingly, a similar Ser-Pro₃ motif is present in CR-A at position 155-158.

Western Analyses with Antiserum to Recombinant CBP86 Indicate Protein Polymorphism and Oligomerization

The cDNA sequence encoding the CBP86 ORF-A was cloned into the bacterial expression vector pET28b and introduced into NovaBlue(DE3) cells. The recombinant protein was purified by immobilized metal affinity chromatography using Ni²⁺- Sepharose. Antiserum against purified rCBP86 was subsequently raised in female rats. Like the rabbit antisera to gel purified CBP86 this monospecific rat antiserum to rCBP86 also recognized multiple protein spots on 2D western blots of human sperm proteins. Immunoreactive species migrated in five major groups based on size: 1) 27-38 kDa; 2) 38-42 kDa; 3) 50-56 kDa; 4) 63-72 kDa; 5)81-87 kDa. The finding of similar patterns of CBP86 isoforms on 2D gels probed with antisera to both the gel purified and recombinant CBP86 confirmed that alternative splice variants identified as cDNAs during cloning were expressed at the protein level resulting in considerable CBP86 heterogeneity. As a further proof of the specificity of the rat and rabbit antisera to CBP86, immunoblots of purified recombinant were probed with the two antisera. Both antisera recognized identical MW forms of the recombinant protein, including high molecular weight complexes >140 kDa, suggestive of oligomerization of the recombinant proteins.

Relationships between the CBP86 isoforms were revealed on 1-D Western blots of SDS extracts of human sperm electrophoresed under reducing conditions where three major immunoreactive forms of CBP86 at 31, 43, and 72 kDa were noted along with several less abundant antigenic bands at 51 and 90-102 kDa. Western blots of non-reduced samples revealed the same abundant 31, 43 and 72 kDa species observed on reduced gels along with prominent immunoreactive bands at 64 and 86 kDa as well as less immunoreactive 34 kDa, 45 kDa, 76 kDa and several higher molecular weight forms. The finding of additional CBP86 forms on nonreduced gels indicated the presence of complexes composed of lower molecular weight forms stabilized by S—S bridges or heterodimerization between LMW CBP86 forms and unknown partner proteins-interactions which had not been fully dissociated by the relatively mild lysis procedure employed for the 2-D gel electrophoresis. Further evidence for CBP86 oligomerization was noted when only one major high molecular weight [HMW] complex was detected on immunoblots obtained from non-reduced native 1-D PAGE gels of human sperm proteins solubilized in 0.2% DOC and 1% NP40 in the absence of reducing agents.

Immunoblotting diagonal gels, in which Laemmli extracts of sperm were analysed by 1D SDS-PAGE in a non-reduced first dimension and then reduced in the second dimension, revealed disaggregation of several high molecular weight CBP86 species. The protein running at 86 kDa on nonreducing gels was shown in the reducing dimension to be comprised of 43 kDa monomers. Similarly, a 76 kDa protein (migrating above the prominent 72 kDa protein on nonreducing gels) appeared to be comprised of 43 kDa and 31 kDa monomers, while the 64 kDa protein was comprised of 31 kDa monomers. The 43 kDa and 31 kDa subunits did not dissociate in the reducing dimension and migrated at the same mass in both reduced and non-reduced 1-D gels. From these immunoblots of diagonal gels it may be concluded that the two major CBP86 forms running on reduced gels at 31 and 43 kDa participate in HMW complexes by both homodimerization and heterodimerization.

The 86 kDa Form of CBP86 Increases with Capacitation

A comparison of extracts from freshly ejaculated human sperm to sperm capacitated in vitro for 5 hours, revealed a substantial increase in the amount of the 86 kDa CBP86 isoforms visible following capacitation. In addition, acidic proteins from groups 2 and 3 of the CBP86 forms (approximate MW 38-42 and 50-56 kDa) were also more prominent in capacitated sperm, including the phosphorylated forms of group 2 previously noted.

Localization of CBP86 in the Seminiferous Epithelium

Immunofluorescent localization of CBP86 in the human testes using the antibody to recombinant CBP86 showed staining of round and elongating spermatids in the seminiferous epithelium and testicular spermatozoa within the lumen of the tubules, indicative of a post-meiotic pattern of expression of the CBP86 gene. The staining patterns suggested a gradual migration of the CBP86 protein from a diffuse cytoplasmic localization in round spermatids to the posterior pole of early spermatids and then to the flagellum as the tail formed. Testes from three patients showed identical localization patterns.

Localization of CBP86 to the Principal Piece of the Mature Human Sperm Flagellum by Immunofluorescence and Immuno-Electron Microscopy

Antibodies raised against rCBP86 recognized the entire length of the principal piece of ejaculated methanol fixed spermatozoa with an intense signal by indirect immunofluorescence microscopy, while both the midpiece and the endpiece exhibited much fainter staining patterns. No CBP86 immunofluorescence was noted in the human sperm head in these non-capacitated sperm. Importantly, no immunofluorescence staining was observed on live motile sperm, indicating that CBP86 epitopes were not accessible on the plasma membrane. A similar staining pattern was achieved with the antiserum raised against gel excised CBP86.

When the distribution of CBP86 in freshly ejaculated human sperm was examined by electron microscopic immunocytochemical staining, gold particles were distributed over the fibrous sheath compartment including the surface of the longitudinal columns and ribs. Smaller numbers of gold partricles were present in the periaxonemal space. CBP86 was not detected in the annular ring or mitochondrial sheath and there was no evidence for CBP86 localization within the axoneme in either the principal piece or distal to the termination of the outer dense fibers.

CBP86 is Tyrosine Phosphorylated During In Vitro Capacitation

Proteins phosphorylated on tyrosine residues during capacitation were identified on 2-D immuno-blots of freshly ejaculated sperm or from sperm capacitated for 3 or 6 hr by staining with the monoclonal anti-phosphotyrosine antibody RC-20. After 3 and 6 hours of in vitro capacitation a significant increase was observed in tyrosine phosphorylation of several sperm proteins including AKAP 3 (fibrous sheath protein 95) and the 64 and 86 kDa forms of CBP86. Following 3 h capacitation the major acidic tyrosine phosphorylated component was a 64 kDa protein. However, after 6 hrs of capacitation the intensity of the 64 kDa protein had diminished and the dominant tyrosine phosphoprotein in the region was the 86 kDa form of CBP86. In addition, a 53 kDa protein showed weak tyrosine phosphorylation after 3 hours of capacitation, while a further increase in phosphorylation of this CBP86 group was observed during the subsequent 3 hours.

Tyrosine phosphorylation of the 86 kDa form of CBP86 varied with the composition of the capacitation medium. Tyrosine phosphorylation of the 86 kDa form of CBP86 in human tubal fluid plus albumin was higher than that observed in Dulbecco's PBS. Interestingly, addition of 100 microM progesterone to the HTF+ albumin containing capacitation media further enhanced the phosphorylation of the most acidic of the CBP86 isoforms. Capacitation-induced tyrosine phosphorylation of the 86 kDa CBP86 isoforms was inhibited in a concentration dependent manner by treatment with the tyrosine kinase inhibitor, genistein, while similar concentrations of the analogue, daidzein had an inhibitory effect on phosphorylation of CBP86 but not FSP 95 (AKAP 3). As a further proof that CBP86 can serve as a substrate for tyrosine kinase, recombinant CBP86 was phosphorylated using an in vitro kinase assay which employed purified baculovirus-expressed c-Src. An increase in tyrosine phosphorylation of CBP86 was noted as its concentration increased in the presence of a constant amount of c-Src, which autophosphorylated as expected. The data confirmed that the acidic tyrosine phosphorylated 86 kDa forms of CBP86 are immunoreactive with anti-rCBP86 and at high resolution the increases in the acidic immunoreactive forms of CBP86 were evident after capacitation in the presence of human tubal fluid and progesterone.

Comparison of ⁴⁵Ca-binding to normal and alkaline phosphatase treated sperm proteins revealed that ⁴⁵Ca readily bound to the acidic (phosphorylated) isoforms of CBP86 in untreated sperm extracts, but in extracts treated with alkaline phosphatase prior to electrophoresis, the calcium binding capacity of the 86 kDa form of CBP86 was abolished, while calcium binding to calreticulin was unaffected. Comparison of the immunoreactive forms of CBP86 present before and after treatment with alkaline phosphatase indicates that alkaline phosphatase reduces the presence of the acidic 86kDa isoforms. Taken together, these observations indicate that both the ⁴⁵Ca binding capacity and the assembly of the 86 kDa form of CBP86 is dependent on phosphorylation.

Discussion

CBP86 is a Highly Polymorphic Protein Derived from Alternatively Spliced Messages and Possibly Translational Readthrough

Six cDNAs for CBP86 have been cloned and sequenced including five alternative splice variants deleting domains of coding regions A or B. On Northern blots broad bands of 2.4 and 1.4 bp were noted indicating polymorphic CBP86 mRNAs. Immunoblotting of reduced and carboxymethylated sperm protein extracts on 1-D gels with antisera to recombinant CBP86 revealed 12 translated forms of CBP86 from 24 to 79 kDa. Isoforms at 67, 59 and 51 kDa were most immunoreactive. Since four CBP86 peptides were found to be larger than the predicted molecular weight (˜53 kDa) from CR-A or splice variants including portions of CR-B (forms VI-VIII and X-XII in FIG. 1) a question is raised about the efficiency of the UAA translation termination signal at the end of CR-A.

The efficiency of translation termination is sensitive to both the 5′ and 3′ sequences adjacent to the stop codon. A strong bias in the relative frequencies of the 3′- flanking bases is found in highly expressed E. coli genes. For UAA the 3′-flanking base preference is U>>G>A>C while for UGA it is U>>A>G>C (Brown et al., 1990). The affinity of release factor for a stop codon is probably enhanced by a specific interaction with the 3′ base (Pedersen et al., 1991). Furthermore, although the last 5 amino acids in proteins are generally not important for protein function, the last 2 amino acids have a cooperative major influence on translation termination (Bjornsson et al., 1996). For the −2 amino acid residue, its acidic/basic as well as hydrophobic/hydrophilic properties are important for termination efficiency. For UAA, the preferred −1 amino acid is a basic residue, lysine in E. coli genes (Brown et al, 1990). In UGA, neutral −2 amino acids provide efficient termination if they have a hydrophilic side chain, and inefficient termination if the side chain is hydrophobic. Examination of the 3′ and 5′ sequences adjacent to UAA in CBP86 revealed that the 3′ adjacent nucleotide (which is G instead of a preferred U), and the 5′ −2 amino acid (which is a hydrophobic residue alanine, instead of a hydrophilic residue) and the 5′ −1 amino acid (which is a acidic residue, glutamic acid instead of a preferred basic residue like lysine) may not favor efficient termination of translation in CR-A of CBP86.

Accordingly, one explanation for the presence of CBP86 peptides larger than those predicted from CR-A or the splice variants (e.g., forms. VI, VII, VIII, X, XI & XII) posits translational readthrough of the UAA termination signal encoded by nucleotides 1528-30. Although stop codon suppression in mammalian cells has been well documented in the translation of different sized proteins from viral genomes such as HIV and MLV (Hatfield et al, 1992) and in the Drosophila kelch gene (Robinson and Cooley, 1997), CBP86 appears to be the first example of translational readthrough of a normal human gene. Previously, translational readthrough was noted in fibroblasts of aspartylglucosaminuria patients with a mutation that creates a premature stop codon in the second exon of the aspartylglucosaminidase [AGA] gene. In this case limited amounts of normally sized AGA was detected. Identification of a normal, endogenous human gene that exhibits translational readthrough may lead to better understanding of both spermiogenesis and HIV susceptibility.

Relationships Between Different Forms of CBP86

Immunoblots of gels run under various conditions revealed oligomerization of CBP86 and relationships between low and high molecular weight forms. When native gels were run and immunoblotted CBP86 migrated as one major high molecular weight complex. On blots of unreduced 1-D SDS-PAGE gels more than 10 distinct immunoreactive CBP86 bands were resolved including prominent bands at 86, 72, 64, 43 and 31 kDa. Reduced but unblocked sperm extracts on 1-D SDS-PAGE gels showed CBP86 immunoreactive forms at 72, 51, 43, and 31 kDa with the 86 kDa and 64 kDa forms present on non-reduced gels no longer apparent. This indicated that the 86 kDa calcium binding form as well as the 64 kDa form were comprised of mercaptoethanol-sensitive subunits. Immunoblot analysis of diagonal gels run in unreduced and then reduced dimensions revealed subunit relationships. One 43 kDa immunoreactive subunit was observed when the 86 kDa form of CBP86 was reduced, suggesting the 86 kDa form consists of homodimers of the protein migrating at 43 kDa or heterodimers between this CBP86 form and an unknown partner(s). Similarly, a 31 kDa immunoreactive form of CBP86 was resolved from reduction of the 64 kDa form suggesting additional homodimerization.

On western blots of 2-D IEF-PAGE gels, five major immunoreactive groups were noted: 1) 27-38 kDa; 2) 38-42 kDa; 3) 50-56 kDa; 4) 63-72 kDa; and 5) 81-87 with each group showing considerable charge heterogeneity ranging overall from pI 4-6. Because the masses deduced from the CBP86 cDNAs differ from the apparent masses of immunoreactive CBP86 forms on gels of reduced and carboxymethylated sperm proteins, post-translational modifications of the proteins encoded by CBP86 splice variants are likely occurring which affect both charge and mass. Antibodies produced to the gel purified 86 KDa spot as well as to rCBP86 show similar immunoreactive groups of proteins on 2-D gels, confirming the heterogeneous nature of the related isoforms and indicating that the alternatively spliced testicular mRNAs which were cloned and sequenced result in translated CBP86 proteins.

Calcium Binds to the Acidic 86 kDa Isoforms of CBP86 which Increase after In Vitro Capacitation

Of the human sperm proteins which bound ⁴⁵calcium only calmodulin gave a stronger signal than the 86 kDa phosphorylated isoforms of CBP86. Although other immunoreactive forms of CBP86 evidence a range of masses and charges on 2-D immunoblots, calcium binding appears to be a property unique to the phosphorylated, oligomerized 86 kDa isoforms with pI of approximately 3.8 to 4.0. Computer analysis did not reveal typical EF-hand motifs or typical poly-acidic stretches in the CBP86 sequence and the calcium binding domain of CBP86 remains unmapped. The oligomerization of the 43 kDa subunit demonstrated by diagonal gel analysis likely relates to the acquisition of calcium binding capacity of the 86 kDa complex.

Increased amounts of the 86 kDa acidic isoforms of CBP86 as well as phosphorylated members of other forms of CBP86 (groups 2 and 3) were observed after in vitro capacitation. Since the 86 kDa CBP86 isoforms consist of 43 kDa immunoreactive subunits, this increase in the 86 kDa isoforms provides the first demonstration, to our knowledge, of a human sperm protein that undergoes oligomerization during capacitation.

Tyrosine Phosphorylation of the Acidic 86 kDa Forms of CBP86 Occurs During In Vitro Capacitation

Increased tyrosine phosphorylation of the acidic 86 kDa and 64 kDa isoforms of CBP86 occurred during capacitation. The 64 kDa forms of CBP86 were prominently phosphorylated after three hours of capacitation. Six hours of capacitation were required for the 86 kDa isoforms to be prominently phosphorylated. The acidic isoforms of CBP86 (pI of 4.0) are the forms that bound calcium⁴⁵ and no calcium binding was observed in the area when the sample was dephosphorylated prior to electrophoretic separation.. It was notable that calreticulin served as a positive control in this experiment since it did not lose calcium binding capability after the sperm extract was treated with alkaline phosphatase prior to electrophoretic separation. Interestingly, treatment of PVDF membranes with alkaline phosphatase after proteins had been transferred did not abolish the calcium binding properties of the 86 kDa CBP86 complex. This suggests that phosphorylation is not essential for calcium binding once the 86 kDa complex has formed.

Together these observations point to a model involving sequential phosphorylation, assembly and acquisition of the calcium binding capacity of the 86 kDa form of CBP86 during the capacitation period. CBP86 represents the first example of calcium binding to a human sperm protein which is tyrosine phosphorylated during capacitation. The characterization of CBP86 adds a third protein substrate to AKAP 4 [originally called AKAP82 or Fsc1 in mouse] and AKAP3 (originally called AKAP95T, FSP95 or AKAP110], which are protein components of the fibrous sheath and were previously shown to be phosphorylated during in vitro capacitation.

Motifs in CBP86

CBP86 possesses putative motifs for self assembly and for progesterone and AKAP binding, and it has an extensin homology. Three of four possible motifs of a SH3 fingerprint are present in the N-terminus of CBP86 and these three SH3 domains are conserved in forms I, VI, VII, VIII, and XII while clones II and XI retain one SH3 domain. Thus, eight of the predicted CBP86 forms have at least one SH3 domain. SH3 domains occur in many proteins and are thought to act as protein binding structures and may be involved in linking signals transmitted from the cell surface by protein tyrosine kinases. The crystal structures of several SH3 domains have been determined, and the view has emerged that SH3 domains facilitate binding to partner proteins by interaction with cognate proline-rich regions containing the consensus sequence Pro-X-X-Pro (PXXP). The three SH3 domains are located at the 5′ end of CR-A of the CBP86 gene, while the 3′ end of CR-A and the 5′ end of the CR-B are relatively proline rich. Three PXXP consensus motifs are present in CR-A [aa 396-399, 471-474, and 473-476) and three are present in CR-B (aa 529-532, 532-535, 643-646), providing structural modules to account for CBP86 self-assembly. Both the SH3 domains and the proline-rich stretches, which we term here the putative dimerization domains, also provide CBP86 with potential sites for interaction with other flagellar proteins (such as the AKAPs).

The domain in CR-A of CBP86 which possesses similarity to the sperm protein SP17 (Richardson et al. 1994) includes amino acids 10-108. Embedded within this SP17 domain at amino acids 10-44 is a region of 40% identity and 57% similarity to the type II regulatory chain of cAMP dependent protein kinase. This homologous region of the mouse and human RII subunits lies in the N-terminus between amino acids 7-41 and contains both the dimerization domain and the AKAP binding region (Newlon et al, 1999). The dimerization domain is the site responsible for the interaction between individual RII subunits while the AKAP binding region, which requires RII dimerization for assembly, mediates binding to A-Kinase Anchor Proteins (AKAPs). The region of RII which is required for high affinity binding to AKAPs is contained within a X-type four-helix bundle dimerization motif with an extended hydrophobic face, a dimerization motif present in another class of signalling molecules, the S100 proteins S100B and calcyclin. By anchoring PK-A to specific regions within a cell, AKAPs direct and specify PK-A action. The RII subunit of PK-A has been demonstrated to bind to two components of the fibrous sheath, AKAP 3 and AKAP 4. AKAP3 localizes to the ribs of the fibrous sheath in human sperm and mouse AKAP 4 has been localized to the ribs and longitudinal columns. The presence of the RII regulatory chain dimerization domain in CBP86 suggests this region of CBP86 may interact with AKAP3 and/or AKAP4 contributing to the supermolecular structure of the fibrous sheath. It may also indicate that CBP86 is phosphorylated by PK-A.

CR-B contains an interesting alignment with the extensin family of hydroxyproline-rich glycoproteins (HRGP's) which are expressed in specific populations of cells in plant roots where they are thought to reinforce the cell wall against mechanical pressure. CBP86 contains two Ser-Pro₃ repeats which resemble the Ser-Pro₄ repeats that characterize the extensins. In view of its structural similarity to the extensins, CBP86's localization throughout the entire principal piece suggests a functional role in strengthening the cytoskeletal framework of the fibrous sheath to resist the mechanical forces of microtubule sliding.

Analysis of the CBP86 sequence detected two motifs of the possible eight of the progesterone receptor. Residues 17 to 102 shared a 20.0% identity with helix domains 9, 10, 11 and 12 of the progesterone receptor binding domain, and a local alignment revealed that 63% of the residues in this region are either identical or conservative replacements. Helix domains 11 and 12 are directly involved with ligand binding. This observation is noteworthy in light of the oligomerization of CBP86 and the possibility that the CBP86 progesterone domains may assemble. Due to CBP86's location in the sperm tail, any progesterone receptor associated with it may be similar to isoform C which lacks the highly conserved DNA binding domain, so a complete match with known progesterone receptor fingerprints is not expected. It is noteworthy that the two progesterone receptor motifs lie within domains of CR-A that undergo alternative splicing, so forms with and without this motif are possible. Progesterone induces the capacitation-related events of hyperactivation and acrosome reaction in human sperm. By stimulating a poorly characterized receptor which appears to differ from nuclear progesterone receptors progesterone triggers a rapid influx of extracellular calcium which results in increased levels of free intracellular calcium followed by phosphatidylinositol 4,5-biphosphate hydrolysis in human sperm. Progesterone induced calcium waves in human sperm are characterized by an initial transient peak followed by a sustained plateau phase lasting for several minutes. Both of these effects depend on extracellular calcium since they do not occur in calcium free medium. Progeterone has also been shown to promote tyrosine phosphorylation of human sperm proteins. It is noteworthy that addition of progesterone to the in vitro capacitation medium leads to phosphorylation of tyrosine residues on the acidic CBP86 isoforms. Progesterone receptors appear as heterodimers (Nishikawa et al, 1995) similar to the hetero polymer complexes formed by CBP86.

### EXAMPLE 2

### Isolation of the Mouse CBP86 Homolog

A mouse testis λTriplEx cDNA library was screened using human CBP85 cDNA as a probe. A positive clone was obtained (named clone 1-7), a 5′ truncated alternatively spliced product which had about 80% similarity to human CBP86-2 and the same splice site. This cDNA clone was then used to screen a mouse Lambda FIX II genomic library.

The mCBP86 genomic insert was about 12 kb in size. The mouse full-length cDNA and genomic sequences of CBP86 had remained unknown so far. For further analysis of mCBP86 genomic clone, it was necessary to get the full-length and alternatively spliced cDNA sequences of mCBP86. The mouse λTriplEx cDNA library was rescreened with a clone 1-7 cDNA probe. 10 positive clones were obtained. The restriction map and DNA sequencing analysis of these clones proved that no full-length cDNA was obtained. The comparison of the cDNA sequences of mCBP86 clone 1- 7 and hCBP86 revealed that there was about 80% similarity between them.

PCR was conducted using a Marathon mouse testis cDNA template and the designed forward primers and 1-7R3, 1-7R2′ reverse primers, and the expected size bands were obtained. Two bands appeared at the PCR product lane amplified with the forward primers and 1-7R2 ′ reverse primer on the agarose gel. The higher band conformed to that of the expected in size, and the lower band was similar in size to the band amplified with the forward primer and 1-7R3 reverse primer. It should be reasonable to suppose that this band is the PCR product of alternatively spliced sequence similar to hCBP86-5.

The sequencing analysis of the PCR products above revealed that at least the four alternatively spliced products of mCBP86 existed in mouse and they had the same splice sites as the corresponding hCBP86 variants. Comparison of cDNA and amino acid sequences between human and mouse CBP86:

1. The cDNA sequences of mouse CBP86 had 78% (CBP86-4) to 81% (CBP86-1 and CBP86-2) similarity and identity with that of human CBP86. For the amino acid sequences, mCBP86-2 had the highest similarity (80%) and identity (76%) with hCBP86; the next to it was mCBP86-4 (similarity 78% and identity 72%). The mCBP86-1 had 72% similarity and 66% identity with hCBP86.

2. The deleted region in mCBP86-2 cDNA sequence from 669 bp (human 590 bp) to 1493 bp (human 1456 bp) of mCBP86-1 had many large gaps compared with the sequence of human CBP86. The sequences before the splice site-668 bp (human 589 bp) and after the splice site-1494 bp (human 1457 bp) were more conserved than the middle region from 669 bp (human 590) to 1493 bp (human 1546 bp) of CBP86.

3. According to the results of the Northern blot, library screening and PCR, it is possible that mCBP86-2 was the most abundant variant of the alternatively spliced products of mCBP86.

4. The mCBP86-1 had calculated MW 48.28 kD and PI 4.48 (hCBP86-1 MW 52.75, PI4.51); the mCBP86-2, MW 41.25 kD and PI 6.55 (hCBP86-2, MW 41 kD, PI 8.65); the mCBP86-3, MW 23.93, PI6.45 (hCBP86-3, MW 23.96, PI 7.68).

5. The comparison of the sequences of the motifSH3, progesterone receptor and CATATPASE displayed that there was high similarity between mCBP86 and hCBP86 except CATATPASE 4 from 61aa to 71aa of hCBP86 which was located on the long gap region of mCBP86.

1. A purified polypeptide comprising
the amino acid sequence of SEQ ID NO: 2;
an amino acid sequence that differs from SEQ ID NO: 2 by one or more conservative amino acid substitutions; or
an amino acid sequence that differs from SEQ ID NO: 2 by a single mutation, wherein the single mutation represents a single amino acid deletion, insertion or substitution.

2. A purified or recombinant polypeptide wherein said polypeptide comprises an amino acid sequence selected from the group consisting of SEQ ID NO: 2SEQ ID NO: 3, SEQ ID NO: 4, SEQ ID NO: 5, SEQ ID NO: 6, SEQ ID NO: 15 and SEQ ID NO: 16.

3. The polypeptide of claim 2 wherein the amino acid sequence is SEQ ID NO: 15 or SEQ ID NO: 16.

4. A nucleic acid sequence comprising the sequence of SEQ ID NO: 1, SEQ ID NO: 25 or SEQ ID NO: 26.

5. A nucleic acid sequence that hybridizes to a 100 nucleotide fragment of SEQ ID NO: 1 under stringent conditions.

6. A transgenic host cell comprising the nucleotide sequence of claim 5.

7. A nucleic acid sequence comprising a 25 bp nucleic acid sequence that is identical to a contiguous 25 bp sequence of SEQ ID NO: 1.

8. A method of screening for potential human therapeutic agents, said method comprising contacting a CBP86 protein with a candidate compound; and determining if the candidate compound selectively binds to the CBP86 protein.

9. The method of claim 8 wherein the CBP86 protein is expressed on the surface of a cell.

10. An antibody that binds specifically to the protein of SEQ ID NO: 2.

11. An antibody that binds specifically to the protein of SEQ ID NO: 15.

12. An antibody that binds specifically to the protein of SEQ ID NO: 16.

13. A method for detecting compounds that inhibit capacitation, said method comprising the steps of
combining a polypeptide comprising an amino acid sequence selected from the group consisting of SEQ ID NO: 2, SEQ ID NO: 3, SEQ ID NO: 4, SEQ ID NO: 5, SEQ ID NO: 6, SEQ ID NO: 15 and SEQ ID NO: 16 with an AKAPs under conditions that allow specific binding of the AKAP to said polypeptide;
contacting said polypeptide with a potential inhibitor; and
measuring the amount of AKAP bound to said polypeptide.

14. A method for measuring the capacitation of sperm in a sample wherein said sample comprises sperm cells containing an 86 kDa isoform of CBP86, said method comprising the step of
measuring the phosphorylation of the 86 kDa isoform.

15. A method for measuring the capacitation of sperm in a sample wherein said sample comprises sperm cells containing an 86 kDa isoform of CBP86, said method comprising the step of
measuring the formation of the 86 kDa isoform of CBP86 relative to other isoforms of CBP86.
